(19) United States
(12) Patent Application Publication
(10) Pub. No.: US 2022/0387393 A1
(43) Pub. Date: Dec. 8, 2022

(54) METHODS FOR THE TREATMENT OF DYSMYELINATING/DEMYELINATING DISEASES

(71) Applicant: Board of Regents, The University of Texas System, Austin, TX (US)

(72) Inventors: Jian HU, Houston, TX (US); Xin ZHOU, Houston, TX (US)

(73) Assignee: Board of Regents, The University of Texas System, Austin, TX (US)

(21) Appl. No.: 17/774,838

(22) PCT Filed: Nov. 6, 2020
(86) PCT No.: PCT/US2020/059272

§ 371 Date: May 5, 2022

Related U.S. Application Data
(60) Provisional application No. 62/931,328, filed on Nov. 6, 2019.

(51) Int. Cl. A61K 31/4184; A61K 31/192; A61P 25/28

(52) U.S. Cl. CPC A61K 31/4184; A61K 31/192; A61P 25/28

(57) ABSTRACT

Provided herein are methods of treating a dysmyelinating/demyelinating disease or condition in a patient in need thereof. The methods comprise restoring Qki-PPARβ-RXRα-dependent lipid metabolism in myelin. For example, the methods comprise administering a PPARβ agonist or an RXR agonist to the patient.

### REFERENCE TO RELATED APPLICATIONS

The present application claims the priority benefit of U.S. provisional application No. 62/931,328, filed Nov. 6, 2019, the entire contents of which is incorporated herein by reference.

### BACKGROUND

### 1. Field

The present invention relates generally to the fields of medicine and neurology. More particularly, it concerns methods of treating dysmyelinating/demyelinating diseases by restoring Qki-PPARβ-RXRα-dependent lipid metabolism in myelin.

### 2. Description of Related Art

Myelin is a specialized multilayer membrane structure that is assembled by oligodendrocytes (OLs) in the central nervous system (CNS) as well as Schwann cells in the peripheral nervous system (PNS). Functionally, the myelin sheath wraps axons to form electrically insulating layers, which enable the rapid salutatory nerve conduction (Siegel, 2006; Armati & Mathey, 2010). Dysmyelinating/demyelinating diseases of central nervous system (CNS) are hereditary and/or acquired disorders in which myelin and/or OLs are injured. Due to the lack of understanding of the disease etiologies, the current treatment options merely alleviate the symptoms. Therefore, there is an urgent clinical need to uncover etiological mechanism-based therapeutic strategies and drug targets against these devastating diseases.

### SUMMARY

In one embodiment, provided herein are methods of treating a dysmyelinating/demyelinating disease or condition, or a disease associated with hypomyelination, in a patient in need thereof, the methods comprising administering a therapeutically effective amount of a PPARβ agonist or an RXR agonist to the patient.

In some aspects, the demyelinating disease is multiple sclerosis. In certain aspects, the multiple sclerosis is progressive multiple sclerosis. In certain aspects, the multiple sclerosis is associated with diffusely abnormal white matter (DAWM). In certain aspects, the patient is maintained on a high fat diet.

In some aspects, the dysmyelinating/demyelinating disease is X-linked adrenoleukodystrophy (X-ALD). In some aspects, the methods further comprise performing hematopoietic bone marrow transplantation on the patient. In some aspects, the demyelinating disease is adrenomyelopathy (AMN).

In some aspects, the demyelinating condition is chemotherapy-induced. In some aspects, the patient has received chemotherapy. In some aspects, the demyelinating condition is chemotherapy-induced cognitive dysfunction.

In some aspects, the methods reduce the severity or delays the onset of one or more neurological deficits in the patient. In some aspects, the methods prolong the survival time of the patient. In some aspects, demyelination of a nerve axon is suppressed. In some aspects, demyelination of a nerve axon is reversed. In some aspects, the methods increase the level of myelin lipids in the patient. In some aspects, the methods restore Qki-PPARβ-RXRα-dependent lipid metabolism in myelin.

In some aspects, the demyelinating disease or condition is a condition of the central nervous system. In some aspects, the PPARβ agonist or RXR agonist is able to cross the blood brain barrier.

In some aspects, the demyelinating disease or condition is a condition of the peripheral nervous system. In some aspects, the PPARβ agonist or RXR agonist is able to penetrate the peripheral nervous system.

In some aspects, the PPARβ agonist is KD3010, GW0742, Telmisartan, L-165041, Elafibranor, or Seladelpar. In some aspects, the RXR agonist is a pan-RXR agonist. In some aspects, the RXR agonist is bexarotene, LG100268, 13-cis retinoic acid (RA), LG101506, or AGN194204. In some aspects, the RXR agonist is a RXRα agonist.

In one embodiment, provided herein are uses of a composition comprising a therapeutically effective amount of a PPARβ agonist or an RXR agonist in the preparation of a medicament for the treatment of a dysmyelinating/demyelinating disease.

In one embodiment, provided herein are compositions comprising a therapeutically effective amount of a PPARβ agonist or an RXR agonist for use in treating a dysmyelinating/demyelinating disease.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G. Lipids are more vulnerable than proteins in PPMS lesions. (FIGS. 1A and 1B) Quantification of immunofluorescent staining of FluoroMyelin (FIG. 1A) and proteolipid protein (PLP) (FIG. 1B) in the white matter from 5 human brains without neurological disease (Control) and 30 lesions from 6 patients with primary progressive multiple sclerosis (PPMS). (FIG. 1C) Scatter plot of FluoroMyelin intensity versus PLP intensity in the samples used in FIG. 1A. The solid lines at 1.0, 1.0 represent the average levels of FluoroMyelin and PLP in the white matter from neurological disease-free human brains. The dashed lines represent 50% of the levels of the solid lines. (FIGS. 1D-1G) Representative images (FIG. 1D) and quantification of FluoroMyelin level (FIG. 1E), PLP level (FIG. 1F), and Olig2+ cell number (FIG. 1G) in neurological disease-free human brains and in 3 subtypes of PPMS lesions, which were divided according to the expression of FluoroMyelin and PLP (FIG. 1C). n=5 in control group; n=3 in Fl^{high} PLP^{low} group; n=15 in Fl^{low} PLP^{high} group; n=12 in Fl^{low} PLP^{low} group. *P<0.05; **P<0.01; ***P<0.001; ****P<0.0001; ns, not significant.

FIGS. 2A-2J. PPARP and RXR agonists alleviate Qki deficiency-induced demyelination. (FIGS. 2A, 2B, 2D, and 2E) The clinical scores and Kaplan-Meier overall survival curves (log-rank test) of Qk-iCKO mice and controls receiving daily oral administration of KD3010, bexarotene (Bex), or vehicle (Veh). The experimental mouse number is indicated in FIGS. 2A and 2D. In FIG. 2A, the four groups within each week represent, from left to right, Ctrl (Veh), iCKO (Veh), Ctrl (KD3010), and iCKO (KD3010). In FIG. 2D, the four groups within each week represent, from left to right, Ctrl (Veh), iCKO (Veh), Ctrl (Bex), and iCKO (Bex). In FIGS. 2B and 2E, the two lines that reach the X-axis represent the iCKO populations. (FIGS. 2C and 2F) Body weights of Qk-iCKO mice and controls receiving daily oral administration of KD3010, Bex, or Veh for 4 weeks after tamoxifen injection. The experimental mouse number is indicated in FIGS. 2C and 2F. (FIGS. 2G and 2H) Representative electron micrographs and quantification of the percentage of myelinated axons and g-ratio of the optic nerves of the experimental mice after 5 weeks of treatment. The experimental mouse number is indicated in FIGS. 2G and 2H. Scale bars, 500 nm. (FIGS. 2I and 2J) Representative images and quantification of FluoroMyelin level in the corpus callosum tissues of experimental mice after 5 weeks of treatment. The experimental mouse number is indicated in FIGS. 2I and 2J. Scale bars, 50 μm. Data are mean±s.d.; one-way ANOVA with Fisher's LSD correction. *P<0.05; **P<0.01; ***P<0.001; ****P<0.0001. ns, not significant.

FIGS. 3A-3B. Qki and PPARβ are major regulators of ABCD2, -3, and -4 expression. (FIG. 3A) CHIP-seq data showing that Qki and PPARβ interact with promoters of ABCD2, -3, and -4. (FIG. 3B) Expressions of ABCD2, -3, and -4 were reduced after Qki depletion in oligodendrocytes.

FIGS. 4A-4C. Chemotherapy-induced demyelination could be alleviated by PPARβ agonist. (FIG. 4A) Quantitative FluoroMyelin staining of the corpus callosum (CC) in 4-month and 6-month mouse brains treated with 5-FU, saline and/or KD3010. (FIG. 4B) Quantitative immunofluorescent staining of oligodendrocyte lineage marker Olig2 in the CC of 6-month mouse brains treated with 5-FU or saline. (FIG. 4C) Quantitative immunofluorescent double staining of Olig2 and Qki in the CC of 6-month mouse brains treated with 5-FU or saline. n.s., not significant.

### DETAILED DESCRIPTION

Dysmyelinating/demyelinating diseases/conditions of central nervous system (CNS) are hereditary and/or acquired disorders in which myelin and/or oligodendrocytes (OLs) are injured. Common demyelinating diseases/conditions include multiple sclerosis (MS), X-linked adrenoleukodystrophy (X-ALD), adrenomyelopathy (AMN), and chemotherapy-induced cognitive dysfunction (chemo brain). Currently there are no effective treatments for these diseases/conditions other than immune modulatory therapies that only alleviate the symptoms but do not treat the diseases themselves. Various forms of dysmyelinating/demyelinating diseases/conditions, including MS, X-ALD, AMN, and chemotherapy-induced demyelination, are due to defective Qki-PPARβ-RXRα-dependent lipid metabolism in myelin. As such, these diseases/conditions can be treated by restoring the function of Qki-PPARβ-RXRα. Therefore, methods of treating these diseases using PPARβ agonists and RXRα agonists are provided.

### I. DYSMYELINATING/DEMYELINATING DISEASES AND CONDITIONS

Myelin is a specialized multilayer membrane structure that is assembled by oligodendrocytes (OLs) in the central nervous system (CNS) as well as Schwann cells in the peripheral nervous system (PNS). Functionally, the myelin sheath wraps axons to form electrically insulating layers, which enable the rapid salutatory nerve conduction (Siegel, 2006; Armati & Mathey, 2010). Dysmyelinating/demyelinating diseases of central nervous system (CNS) are hereditary and/or acquired disorders in which myelin and/or OLs are injured. Exemplary dysmyelinating/demyelinating disease and conditions include: leukodystrophy; leukoencephalopathy; central pontine myelinolysis; Alexander disease; Canavan disease; globoid cell leukodystrophy (Krabbe disease); megalencephalic leukoencephalopathy with subcortical cysts; metachromatic leukodystrophy; Pelizaeus-Merzbacher disease; vanishing white matter disease; x-linked adrenoleukodystrophy; Aicardi-Goutières syndrome; CIC-2-related disease; giant axonal neuropathy; megalencephalic leukoencephalopathy with subcortical cysts; oculodentodigital dysplasia; vanishing white matter disease; early-onset neuronal degenerative disorders; GM1 & GM2 gangliosidoses (e.g. Tay Sachs disease); giant axonal neuropathy; hypomyelination with atrophy of basal ganglia and cerebellum (H-ABC); hypomyelination with congenital cataract; leukoencephalopathy with brainstem and spinal cord involvement and lactate elevation; hypomyelination with brainstem and spinal cord involvement and leg spasticity; Pol III-related leukodystrophies; cerebral autosomal dominant arteriopathy with subcortical infarcts and leukoencephalopathy; cerebral autosomal recessive arteriopathy with subcortical infarcts and leukoencephalopathy; cathepsin A-related arteriopathy with strokes and leukoencephalopathy; cerebral amyloid angiopathy; Fabry disease; leukoencephalopathy with calcifications and cysts (Labrun syndrome); CSF1R-related disorders; adult-onset leukoencephalopathy with axonal spheroids and pigmented glia; pigmentary ortochromatic leukodystrophy; Nasu Hakola disease; 18q-deletion syndrome; fucosidosis; Pelizaeus-Merzbacher disease; Pelizaeus-Merzbacher-like disease 1; globoid cell leukodystrophy (Krabbe disease); Kearns-Sayre syndrome; Leigh disease; mitochondrial encephalomyopathy, lactic acidosis, and stroke-like episodes (MELAS); myoclonic epilepsy with ragged red fibers (MERRF); multiple sulfatase deficiency; phenylketonuria; adult-onset autosomal dominant leukodystrophy; cerebrotendinous xanthomathosis; cystic leukoencephalopathy without megalencephaly; L-2-hydroxyglutaric aciduria; lysosomal storage diseases; free sialic acid storage disorders (e.g. Salla disease); Niemann-Pick disease; peroxisomal disorders; Zellweger syndrome; Sjögren-Larsson syndrome; multiple sclerosis (MS); Marburg variant of multiple sclerosis; Schilder type multiple sclerosis; concentric sclerosis of Balo; tumefactive demyelinating lesion; neuromyelitis optica (NMO); central pontine myelinolysis; acute disseminated encephalomyeltitis (ADEM); CNS neuropathies induced by vitamin B12 deficiency; anti-MOG associated encephalomyelitis; acute hemorrhagic leukoencephalitis (Weston Hurst syndrome); Guillain-Barré syndrome (GBS); Charcot-Marie-Tooth disease; copper deficiency-associated conditions; progressive inflammatory neuropathy; chronic inflammatory demyelinating polyneuropathy (CIDP); idiopathic inflammatory demyelinating diseases; POEMS syndrome; transverse myelitis; radiologically isolated syndrome (RIS); viral demyelination; progressive multifocal leucoencephalopathy (PML); multiple system atrophy (MSA); Med12 related disorders; FG syndrome; Lujan syndrome; the X-linked recessive form of Ohdo syndrome; adenosine deaminase deficiency (ADA deficiency); HIV related white matter changes; HIV encephalitis; subacute sclerosing panencephalitis (SSPE); toxic or metabolic demyelination; osmotic demyelination; toxic leukoencephalopathy; vascular (small-vessel disease) demyelination; small vessel cerebral ischemia; chronic hypertensive encephalopathy; cerebral amyloid angiopathy; trigeminal neuralgia; chemotherapy-induced demyelination and cognitive dysfunction; radiotherapy-induced demyelination and cognitive dysfunction; and spinal cord injury. Exemplary hypomyelination diseases include: memory disorders; Parkinson's disease; peripheral neuropathy; post-herpetic neuralgia; spinal cord tumor; stroke; amyotrophic lateral sclerosis (ALS); arteriovenous malformation; brain aneurysm; brain tumors; dural arteriovenous fistulae; epilepsy; headache; schizophrenia; Alzheimer's disease; Huntington's disease; and motor neuron disease.

Multiple sclerosis (MS) is the most common demyelinating disease that affects approximately 400,000 people in the United States and 2.5 million people worldwide (Noseworthy et al., 2000). MS exhibits great variability in genetics, clinical courses, magnetic resonance imaging, pathology, and responses to therapies among/within patients, suggesting its multi-etiological nature (Lucchinetti et al., 2000; Lassmann et al., 2001). The etiology of MS is unclear, although many people believe that it is principally an autoimmune disease in which the dysregulated immune system primarily attacks OLs and/or myelin sheath, leading to the subsequent demyelination and inflammation (McFarland & Martin, 2007; Stys et al., 2012). However, the mechanism that arouses immune autoreactivity is not known, and no consistent myelin antigens have been identified (Filippi et al., 1998; Traka et al., 2016). In addition, conflicting with the autoimmune hypothesis, which implies a major role of OL cell death in MS, extensive studies showed that a large proportion of MS patients (30-60%) exhibit no apparent OL cell death, suggesting OL cell death-independent pathogenic mechanisms of MS (Lucchinetti et al., 2000; Lassmann et al., 2001; Lucchinetti et al., 1996; Lucchinetti et al., 1999; Raine et al., 1981; Bruck et al., 1994). Currently, there are two types of treatment for MS: immunomodulatory therapy for the associated immune disorders and therapies to relieve or modify symptoms. However, due to the lack of understanding of the disease etiologies, these treatment options can only relieve or slow the progression of the symptoms but not cure the disease itself. Therefore, there is an urgent clinical need to uncover etiological mechanism-based therapeutic strategies and drug targets against this devastating disease.

X-linked adrenoleukodystrophy (X-ALD) is one of the most common leukodystrophies with devastating outcomes in both children and adults, and yet since its description decades ago, effective treatments with clear mechanisms remain wanting. Pathognomonic to these patients is the accumulation of saturated very long chain fatty acids (VLCFAs) in tissues and blood, attributed to mutations in a peroxisomal membrane half-transporter called ATP-binding cassette sub-family D member 1 (ABCD1) (Berger et al., 2014; Waldman, 2018). A key characteristic of X-linked adrenoleukodystrophy (X-ALD) is white matter degeneration in the central nervous system (CNS) (Waldman, 2018; van der Knapp & Bugiani, 2017). Between 30% and 40% of male patients experience progressive cerebral demyelination in childhood or adolescence resulting in dementia and a 5-year survival rate of 66% (Waldman, 2018; Mahmood et al., 2007) The remaining patients develop a debilitating spinal cord disease known as adrenomyelopathy (AMN), with risk of progressing to cerebral involvement as well (Waldman, 2018). Hematopoietic bone marrow transplantation (HSCT) has been shown to benefit a subset of patients, mainly children with early cerebral disease (Miller et al., 2011; Peters et al., 2004). However, after HSCT it can take several months before the demyelination stops progressing, during which time irreversible cognitive and sensory-motor damage still occurs. Moreover, HSCT has no disease modifying effects in children with moderate to severe symptoms or in AMN patients.

Chemotherapy-induced neurological sequelae (commonly called chemo brain), which lack effective treatment, affect up to 75% of cancer patients, diminishing these individuals' quality of life and complicating their care (Stewart et al., 2008; Silverman et al., 2007; Schagen et al., 1999; Meyers & Abbruzzese, 1992; Kreukels et al., 2005; Jenkins et al., 2006; Jenkins et al., 2006; Inagaki et al., 2007; Hurria et al., 2006; Fan et al., 2005; Falleti et al., 2005; Downie et al., 2006; Bender et al., 2006; Kovalchuk & Kolb, 2017; Bougea et al., 2016; Janelsins eta 1., 2018). For instance, 30%-75% of breast cancer patients have cognitive deficits after chemotherapy, and these adverse neurological sequelae (commonly called chemo brain) can last 2-10 years even after treatment cessation (Schagen et al., 1999; Meyers & Abbruzzese, 1992; Stemmer et al., 1994; Brown et al., 1998). Interestingly, adverse neurological sequelae do not seem to be associated with specific types of chemotherapeutic agents, most of which do not penetrate brain well. For instance, adverse neurological sequelae have been observed with antimetabolites (such as cytosine arabinoside (Poppelreuter et al., 2004)), 5-fluorouracil (5-FU) (Choi et al., 2001; Johnson et al., 1999), methotrexate (Haykin et al., 2006; Shuper et al., 2000; Shuper et al., 2002), DNA cross-linking agents (such as BCNU (Kleinschmidt-DeMasters, 1986) and cisplatin (Bruck et al., 1989)), and, intriguingly, antihormonal agents (Silverman et al., 2007; Eberling et al., 2004; Paganini-Hill & Clark, 2000; Shilling et al., 2003; Castellon et al., 2005). The typical adverse neurological sequelae associated with chemotherapy include leukoencephalopathy, seizures, cerebral infarctions, and cognitive dysfunction (Dietrich, 2010; Janelsins et al., 2011). The cause(s) of these adverse neurological sequelae are not clear; however, demyelination seems to be the most consistent neurological change and could explain all these sequelae.

### II. PPARβ AGONISTS AND RXR AGONISTS

A peroxisome proliferator activated receptor-beta (PPARβ) agonist is a fatty acid, lipid, protein, peptide, small molecule, or other chemical entity that binds to the cellular PPARβ and elicits a downstream response, namely gene transcription, either native gene transcription or a reporter construct gene transcription, comparable to endogenous ligands such as retinoic acid or comparable to a standard reference PPARβ agonist such as carbacyclin.

In an embodiment, a PPARβ agonist is a selective agonist. As used herein, a selective PPARβ agonist is viewed as a chemical entity that binds to and activates the cellular PPARβ and does not substantially activate the cellular peroxisome proliferator activated receptors-alpha (PPARα) and -gamma (PPARγ). As used herein, a selective PPARβ agonist is a chemical entity that has at least a 10-fold maximum activation (as compared to endogenous receptor ligand) with a greater than 100-fold potency for activation of PPARβ relative to either or both of PPARα and PPARγ. In a further embodiment, a selective PPARβ agonist is a chemical entity that binds to and activates the cellular human PPARβ and does not substantially activate either or both of human PPARα and PPARγ. In a further embodiment, a selective PPARβ agonist is a chemical entity that has at least a 10 fold, or a 20 fold, or a 30 fold, or a 40 fold, or a 50 fold, or a 100 fold potency for activation of PPARβ relative to either or both of PPARα and PPARγ.

“Activation” here is defined as the abovementioned downstream response, which in the case of PPAR's is gene transcription. Gene transcription may be measured indirectly as downstream production of proteins reflective of the activation of the particular PPAR subtype under study. Alternatively, an artificial reporter construct may be employed to study the activation of the individual PPAR's expressed in cells. The ligand binding domain of the particular receptor to be studied may be fused to the DNA binding domain of a transcription factor, which produces convenient laboratory readouts, such as the yeast GAL4 transcription factor DNA binding domain. The fusion protein may be transfected into a laboratory cell line along with a Gal4 enhancer, which effects the expression of the luciferase protein. When such a system is transfected into a laboratory cell line, binding of a receptor agonist to the fusion protein will result in light emission.

A selective PPARβ agonist may exemplify the above gene transcription profile in cells selectively expressing PPARβ, and not in cells selectively expressing PPARγ or PPARα. In an embodiment, the cells may be expressing human PPARβ, PPARγ, and PPARα, respectively.

In a further embodiment, a PPARβ agonist may have an EC50 value of less than 5 m as determined by the PPAR transient transactivation assay described below. In an embodiment, the EC50 value is less than 1 m. In another embodiment, the EC50 value is less than 500 nM. In another embodiment, the EC50 value is less than 100 nM. In another embodiment, the EC50 value is less than 50 nM.

In an embodiment, a PPARβ agonist may be a PPARβ agonist compound as disclosed in any of the following published patent applications: WO 97/027847, WO 97/027857, WO 97/028115, WO 97/028137, WO 97/028149, WO 98/027974, WO 99/004815, WO 2001/000603, WO 2001/025181, WO 2001/025226, WO 2001/034200, WO 2001/060807, WO 2001/079197, WO 2002/014291, WO 2002/028434, WO 2002/046154, WO 2002/050048, WO 2002/059098, WO 2002/062774, WO 2002/070011, WO 2002/076957, WO 2003/016291, WO 2003/024395, WO 2003/033493, WO 2003/035603, WO 2003/072100, WO 2003/074050, WO 2003/074051, WO 2003/074052, WO 2003/074495, WO 2003/084916, WO 2003/097607, WO 2004/000315, WO 2004/000762, WO 2004/005253, WO 2004/037776, WO 2004/060871, WO 2004/063165, WO 2004/063166, WO 2004/073606, WO 2004/080943, WO 2004/080947, WO 2004/092117, WO 2004/092130, WO 2004/093879, WO 2005/060958, WO 2005/097098, WO 2005/097762, WO 2005/097763, WO 2005/115383, WO 2006/055187, WO 2007/003581, WO 2007/071766, and WO 2015/035171.

In another embodiment, a PPARβ agonist may be a compound selected from the group consisting of sodelglitazar; lobeglitazone; netoglitazone; and isaglitazone; 2-[2-methyl-4-[[3-methyl-4-[[4-(trifluoromethyl)phenyl]methoxy]phenyl]thio]phenoxy]-acetic acid (See WO 2003/024395); (S)-4-[cis-2,6-dimethyl-4-(4-trifluoromethoxy-phenyl)piperazine-1-sulfonyl]-indan-2-carboxylic acid or a tosylate salt thereof (KD-3010); 4-butoxy-a-ethyl-3 -[[[2-fluoro-4-(trifluoromethyl)benzoyl]amino]methyl]-benzenepropanoic acid (TIPP-204); 2-[2-methyl-4-[[[4-methyl-2-[4-(trifluoromethyl)phenyl]-5-thiazolyl]methyl]thio]phenoxy]-acetic acid (GW-501516); 2-[2,6 dimethyl-4-[3-[4-(methylthio)phenyl]-3-oxo-1(E)-propenyl]phenoxyl]-2-methylpropanoic acid (GFT-505); and {2-methyl-4-[5-methyl-2-(4-trifluoromethyl-phenyl)-2H-[1,2,3]triazol-4-ylmethylsylfanyl]-phenoxy}-acetic acid.

The Retinoic Acid Receptors (RARs) and Retinoid X Receptors (RXRs) and their cognate ligands function by distinct mechanisms. The RARs always form heterodimers with RXRs and these RAR/RXR heterodimers bind to specific response elements in the promoter regions of target genes. The binding of RAR agonists to the RAR receptor of the heterodimer results in activation of transcription of target genes leading to retinoid effects. On the other hand, RXR agonists do not activate RAR/RXR heterodimers. RXR heterodimer complexes like RAR/RXR can be referred to as non-permissive RXR heterodimers as activation of transcription due to ligand-binding occurs only at the non-RXR protein (e.g., RAR); activation of transcription due to ligand binding does not occur at the RXR. RXRs also interact with nuclear receptors other than RARs and RXR agonists may elicit some of its biological effects by binding to such RXR/receptor complexes. These RXR/receptor complexes can be referred to as permissive RXR heterodimers as activation of transcription due to ligand-binding could occur at the RXR, the other receptor, or both receptors. Examples of permissive RXR heterodimers include, without limitation, peroxisome proliferator activated receptor/RXR (PPAR/RXR), farnesyl X receptor/RXR (FXR/RXR), nuclear receptor related-1 protein (Nurr1/RXR) and liver X receptor/RXR (LXR/RXR). Alternately, RXRs may form RXR/RXR homodimers which can be activated by RXR agonists leading to rexinoid effects. Also, RXRs interact with proteins other than nuclear receptors and ligand binding to an RXR within such protein complexes can also lead to rexinoid effects. Due to these differences in mechanisms of action, RXR agonists and RAR agonists elicit distinct biological outcomes and even in the instances where they mediate similar biological effects, they do so by different mechanisms. Moreover, the unwanted side effects of retinoids, such as pro-inflammatory responses or mucocutaneous toxicity, are mediated by activation of one or more of the RAR receptor subtypes. Stated another way, biological effects mediated via RXR pathways would not induce pro-inflammatory responses, and thus, would not result in unwanted side effects.

Thus, aspects of the present specification provide, in part, an RXR agonist. As used herein, the term “RXR agonist” refers to a compound that binds to one or more RXR receptors, like an RXRα, in a manner that elicits gene transcription via an RXR response element. As use the term “selective RXR agonist” refers to the discriminatory activation of heterodimeric partners for RXR (such as a PPAR or a LXR) upon binding of a RXR agonist to one type of RXR heterodimer but not to another.

In one embodiment, the RXR agonist activates the permissive heterodimer PPAR/RXR, but not other permissive RXR heterodimers. Examples of an RXR agonist which activates PPAR/RXR include, e.g., LGD1069 (bexarotene), LGD268, AGN194204.

### III. METHODS OF TREATMENT

The present invention is generally directed to methods of treating demyelination in a subject in need thereof comprising administering to the subject an effective amount of a PPARβ agonist or RXR agonist.

As used herein, “administer” or “administering” means to introduce, such as to introduce to a subject a compound(s) or composition. The term is not limited to any specific mode of delivery, and can include, but is not limited to, transdermal and oral delivery.

As used herein, “treat” or “treating” or “treatment” can refer to one or more of: delaying the progress of a disorder; controlling a disorder; delaying the onset of a disorder; ameliorating one or more symptoms characteristic of a disorder; or delaying the recurrence of a disorder, or characteristic symptoms thereof, depending on the nature of the disorder and its characteristic symptoms.

As used herein, “subject” or “patient” generally refers to a human, but also may include other mammals such as horses, cows, sheep, pigs, mice, rats, dogs, cats, and primates. In an embodiment, the subject is a human. In another embodiment, the subject is a mammal who exhibits one or more symptoms characteristic of a disorder. In another embodiment, the subject is a human who exhibits one or more symptoms characteristic of a disorder. The term subject does not require one to have any particular status or relationship with respect to a hospital, clinic, research facility, or physician (e.g., as an admitted patient, a study participant, or the like).

Dosages of the compounds used in the present invention must ultimately be set by an attending physician. General outlines of the dosages are provided herein below. Generally, a suitable dose of a PPARβ agonist or RXR agonist, or a pharmaceutically acceptable salt thereof, for administration to a human will be in the range of about 0.1 mg/kg per day to about 25 mg/kg per day (e.g., about 0.2 mg/kg per day, about 0.3 mg/kg per day, about 0.4 mg/kg per day, about 0.5 mg/kg per day, about 0.6 mg/kg per day, about 0.7 mg/kg per day, about 0.8 mg/kg per day, about 0.9 mg/kg per day, about 1 mg/kg per day, about 2 mg/kg per day, about 3 mg/kg per day, about 4 mg/kg per day, about 5 mg/kg per day, about 6 mg/kg per day, about 7 mg/kg per day, about 8 mg/kg per day, about 9 mg/kg per day, about 10 mg/kg per day, about 15 mg/kg per day, about 20 mg/kg per day, or about 25 mg/kg per day). Alternatively, a suitable dose of a PPARβ agonist or RXR agonist, or a pharmaceutically acceptable salt thereof, for administration to a human will be in the range of from about 0.1 mg/day to about 1000 mg/day; from about 1 mg/day to about 400 mg/day; or from about 1 mg/day to about 300 mg/day. In other embodiments, a suitable dose of a PPARβ agonist or RXR agonist, or a pharmaceutically acceptable salt thereof, for administration to a human will be about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, or about 500 mg/day. Dosages may be administered more than one time per day (e.g., two, three, four, or more times per day). In one embodiment, a suitable dose of a PPARβ agonist or RXR agonist, or a pharmaceutically acceptable salt thereof, for administration to a human is about 100 mg twice/day (i.e., a total of about 200 mg/day). In another embodiment, a suitable dose of a PPARβ agonist or RXR agonist, or a pharmaceutically acceptable salt thereof, for administration to a human is about 50 mg twice/day (i.e., a total of about 100 mg/day).

In some aspects of the invention, PPARβ agonist or RXR agonist is administered in a therapeutically effective amount to a subject (e.g., a human). As used herein, the term “effective amount” or “therapeutically effective amount” refers to an amount of an active ingredient that elicits the desired biological or medicinal response, for example, reduction or alleviation of the symptoms of the condition being treated. In some embodiments of the invention, the amount of PPARβ agonist or RXR agonist administered can vary depending on various factors, including, but not limited to, the weight of the subject, the nature and/or extent of the subject's condition, etc.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Activating Qki-PPARβ-RXRα Treats MS

Myelin has long been considered as an inert material (Uzman & Hedley-Whyte, 1968; Smith, 1968), while more recent studies imply that continuous supply of myelin structural components happens during adulthood (Ando et al., 2003; Marcus et al., 2006). Although the unique structure and composition of myelin are static, gradual turnover of myelin components is believed to be essential for maintaining the integrity of this characteristic membrane and fulfill its insulating function (Ando et al., 2003). The dry mass of myelin comprises 30% proteins and 70% lipids in the CNS (Siegel, 2006; Armati & Mathey, 2010). Among them, the major myelin proteins and cholesterol belong to the slow exchanging pool (Smith, 1968; Ando et al., 2003; Price et al., 2010; Toyama et al., 2013), while the phospholipids belong to the rapid exchanging pool (Ando et al., 2003; Morell & Ousley, 1994; Smith & Eng, 1965). These observations indicate that myelin lipids are probably more vulnerable to intrinsic or extrinsic insults than myelin proteins during a lifetime. Supporting the importance of lipid metabolism in MS pathogenesis, several recent studies showed that in a special subtype of MS, Diffusely Abnormal White Matter (DAWM), myelin lipid level was greatly reduced yet the myelin protein level was relatively unchanged (Moore et al., 2008; Laule et al., 2013), indicating that myelin lipid metabolism may play an important pathogenic role in MS.

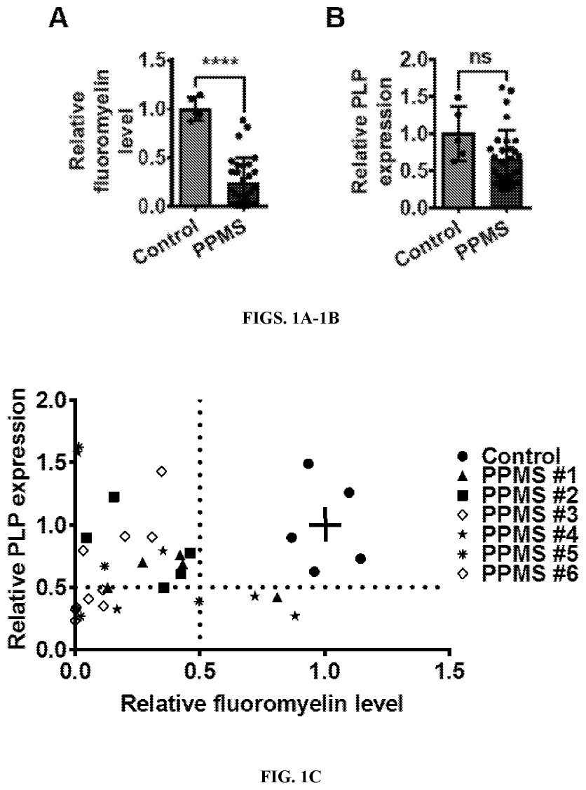

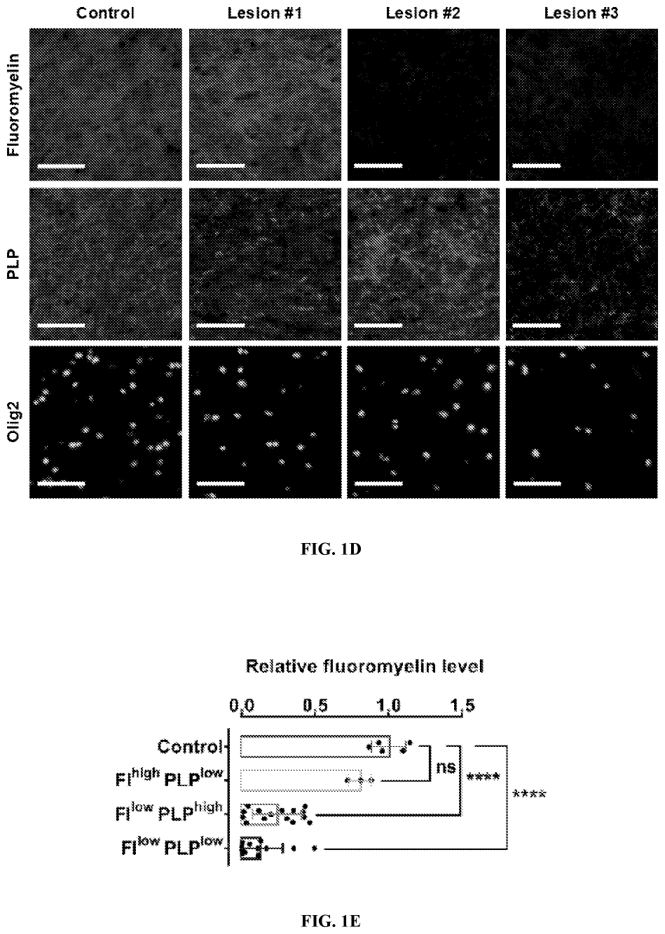

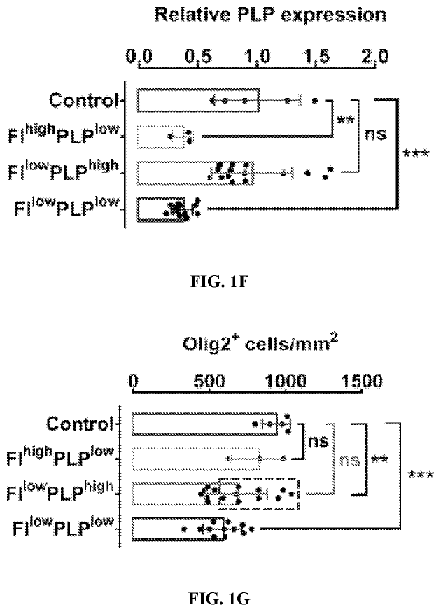

To investigate the potential role of myelin lipid regulation in the pathogenesis of human demyelinating diseases, whether myelin lipid levels were preferentially reduced relative to levels of the protein components or overall oligodendrocyte numbers in human MS lesions was determined. Postmortem brain slices of 6 patients with primary progressive MS (PPMS) and of 5 control donors without neurological disease were obtained from the Rocky Mountain MS Center. Thirty individual chronic lesions were identified in the 6 specimens from patients with PPMS. All the samples were characterized by local pathologists before enrollment in the tissue bank (Popescu et al., 2013). The average level of myelin lipids in these MS lesions, as measured by FluoroMyelin staining, was 24.8% of that detected in the white matter from the control brain slices (FIG. 1A). In contrast, despite marked variations among individual lesions, the average levels of the major myelin structural proteins (PLP, MBP, and MAG) were not significantly different in the MS lesions and the control white matter (FIG. 1B). By grouping the MS lesions on the basis of their myelin lipid and protein levels, it was found that 50% (15/30) of the lesions exhibited low lipid levels but normal protein levels (Fl^{low}PLP^{high}), 10% (3/30) of the lesions exhibited normal lipid levels but low protein levels (Fl^{high}PLP^{low}), and the remaining 40% (12/30) exhibited low levels of both lipids and proteins (Fl^{low}PLP^{low})(FIGS. 1C-1F). These data suggest that myelin lipids are preferentially reduced in a subset of PPMS lesions.

Oligodendrocyte death is often considered a primary pathogenic event in MS (Traka et al., 2016). To determine whether the observed lower levels of myelin lipids in MS lesions correlated with a lower density of oligodendrocytes, the number of Olig2+ oligodendrocytes in each MS lesion were counted. Fl^{low}PLP^{low} lesions had significantly lower numbers of oligodendrocytes than did control brain tissue (P<0.001), suggesting that oligodendrocyte death was likely the major cause of the demyelination and decreased levels of myelin lipids and proteins observed in this subgroup of lesions (FIGS. 1D and 1G). In contrast, 60% (9/15) of the Fl^{low}PLP^{high} lesions (indicated by the red dashed box in FIG. 1G) had normal oligodendrocyte densities, suggesting that diminished myelin lipid levels, rather than oligodendrocyte death, might be responsible for demyelination in this subgroup of lesions (FIGS. 1D and 1G). The remaining 40% (6/15) of the Fl^{low}PLP^{high} lesions had relatively low oligodendrocyte density but normal myelin protein staining. One explanation for this discrepancy is that the myelin proteins released by dead oligodendrocytes had yet to be degraded owing to their long half-life (Price et al., 2010). Notably, some lesions from the same patients were heterogeneous with respect to myelin protein levels, myelin lipid levels, and oligodendrocyte density, indicating that even in an individual patient, different lesions may be caused by distinct pathological events (FIG. 1C).

To determine whether the preferential reduction in myelin lipids in MS lesions is due to compromised functionality of the Qki-PPARβ-RXRα complex, the expression levels of the genes that are involved in lipid metabolism, normalized to PLP expression levels, in chronic MS lesions and the white matter of neurological disease-free human brains were compared (Han et al., 2012). Gene set enrichment analysis of eight Qki-PPARβ-RXRα-associated lipid metabolism pathways, including fatty acid metabolism, fatty acid biosynthesis, biosynthesis of unsaturated fatty acids, fatty acid elongation, fatty acid degradation, PPAR signaling, glycerophospholipid metabolism, and sphingolipid metabolism, revealed that the activities of these pathways were significantly downregulated in the MS lesions relative to the control white matter. Notably, the levels of SCD, SCD5, HACDl, ELOVLl, and ELOVL5, the known Qki-PPARβ-RXRα target genes that encode key enzymes for fatty acid desaturation and elongation, were all significantly lower in the MS lesions than in the control white matter. The transcriptional downregulation of genes and pathways involved in Qki-PPARβ-RXRα-associated lipid metabolism, together with the observation that a subset of MS lesions exhibits preferential reduction in lipid levels, suggest that disruption of lipid homeostasis plays an important role during pathological demyelination in MS. Supporting the importance of lipid metabolism in MS pathogenesis, several recent studies showed that in diffusely abnormal white matter (DAWM), a subtype of MS, myelin lipid levels were greatly reduced yet myelin protein levels and oligodendrocyte numbers were relatively unchanged (Moore et al., 2008; Laule et al., 2013), reinforcing the idea that myelin lipid metabolism may play an important pathogenic role in MS.

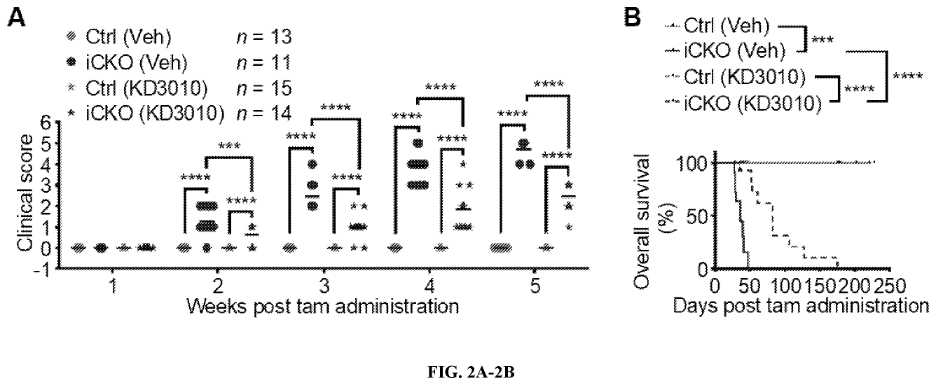

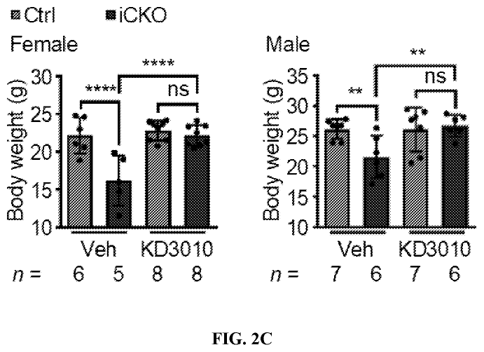

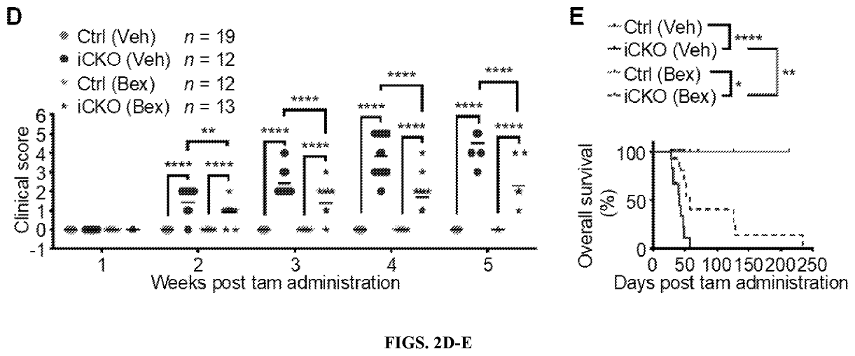

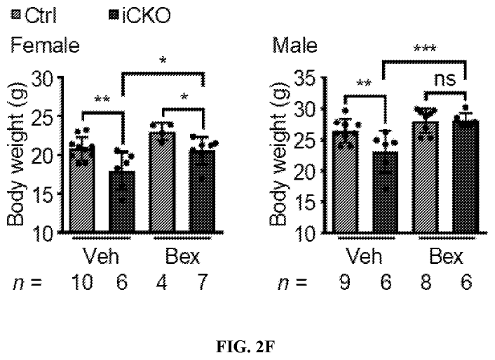

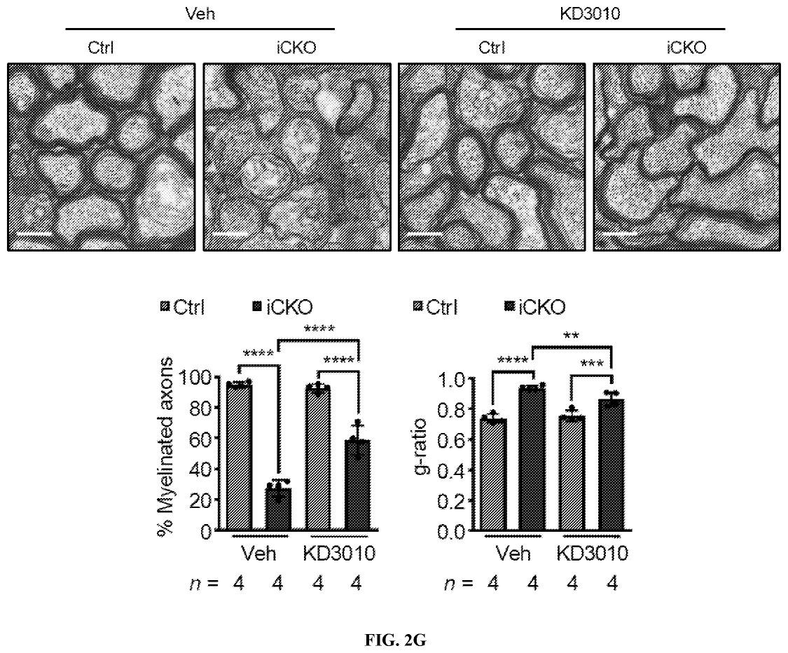

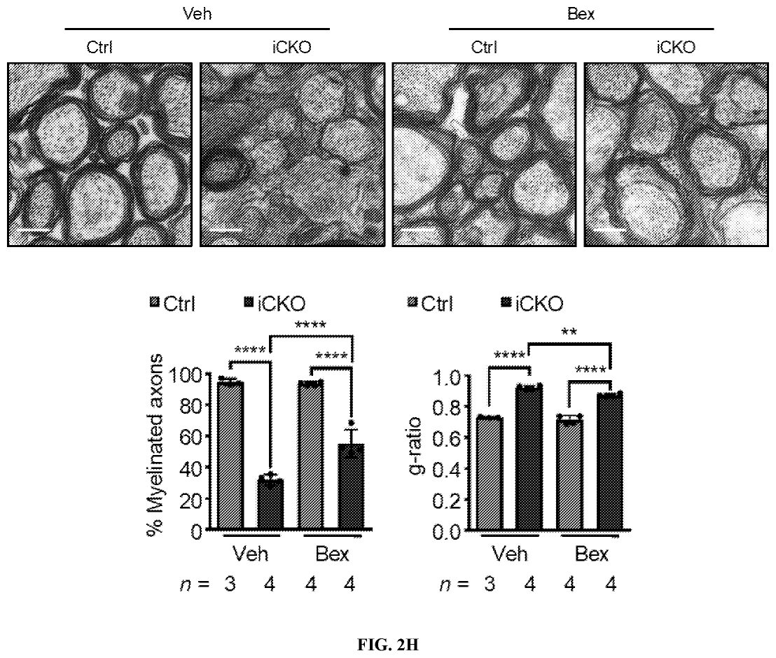

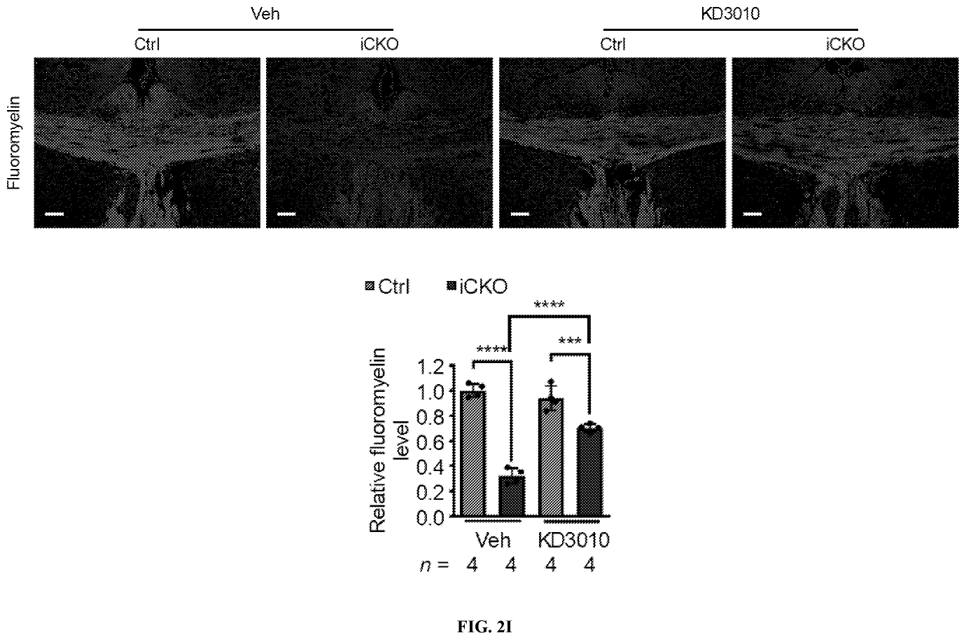

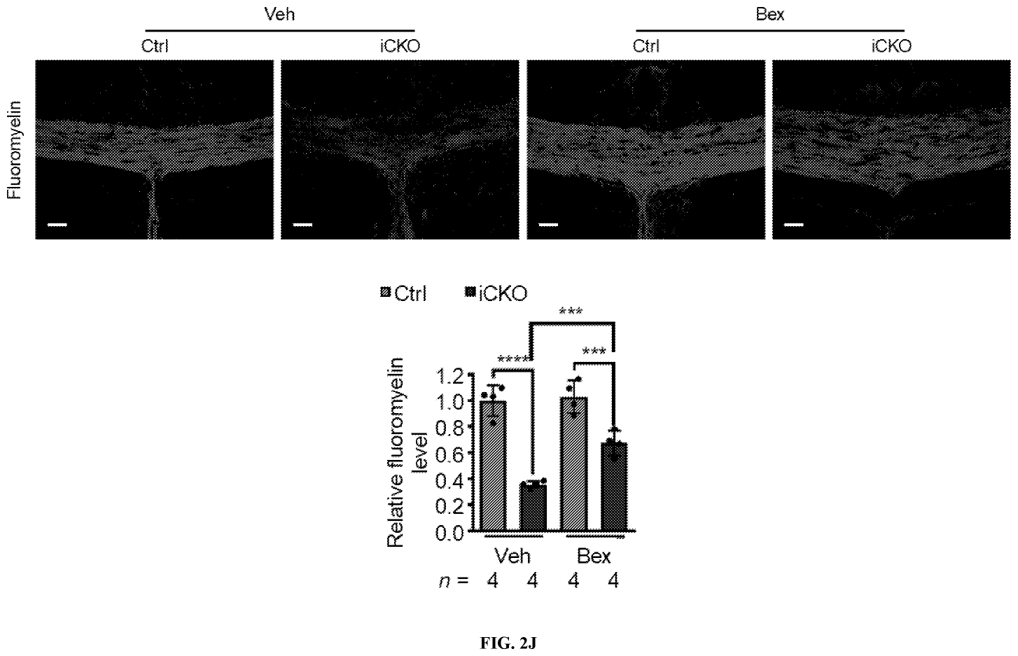

These findings indicated that Qki is a coactivator of PPARβ-RXRα and that its loss diminishes, but does not totally abolish, PPARβ-RXRα transcriptional activity. Therefore, whether PPARβ-RXRα agonists could alleviate lipid defect-induced demyelination and its related symptoms was tested. First, it was determined whether the CNS-permeable PPARβ agonist KD3010 (Dickey et al., 2016) and RXR agonist bexarotene (Cramer et al., 2012) could alleviate Qki depletion-induced demyelinating symptoms. Oral administration of KD3010 or bexarotene in mice with Qk knockout in mature oligodendrocyte (Qk-iCKO mice) significantly delayed the onset of neurological deficits, lowered clinical scores, increased body weights, and prolonged median survival times (2.34- or 1.41-fold, respectively) compared to treatment with vehicle (FIGS. 2A-2F). Strikingly, 23.1% (3 of 13) of Qk-iCKO mice treated with KD3010 or bexarotene lived for more than 100 days, with the maximum survival duration increased by 3.72- or 4.03-fold compared to controls, respectively (FIGS. 2B and 2E). Consistent with their clinical improvement, Qk-iCKO mice treated with KD3010 or bexarotene had 115.3% or 73.7% more myelinated axons, with thicker myelin sheaths, than did Qk-iCKO mice treated with vehicle (FIGS. 2G and 2H). In support of this, Qk-iCKO mice treated with KD3010 or bexarotene had 119.4% or 89.5% higher myelin lipid levels than did Qk-iCKO mice treated with vehicle (FIGS. 2I and 2J). These significant alleviations of Qki depletion-induced demyelinating symptoms by PPARβ and RXR agonists confirm that PPARβ-RXRα-dependent fatty acid metabolism is a key downstream target of Qki in maintaining mature myelin homeostasis and provide justification for the use of PPARβ-RXRα-agonists in future clinical trials for patients with demyelinating diseases. Also, like bexarotene and KD3010, a high-fat diet alleviated the demyelinating phenotypes in Qki-depleted mice (FIG. 2D), further supporting the idea that Qki-PPARβ-RXRα is the major pathway regulating lipid metabolism in mature myelin. The observation that the RXR agonist and high-fat diet significantly alleviated Qki depletion-induced demyelinating symptoms confirmed that Qki-PPARβ-RXRα-dependent fatty acid metabolism is a major pathway that maintains the lipid pool of mature myelin, and activating this pathway can help alleviate demyelination in MS patients, particularly the progressive form of MS.

### Example 2

### Activating Qki-PPARβ-RXRα Treats X-ALD and AMN

X-ALD arises from mutations in the ATP-binding cassette subfamily D member 1 (ABCD1) gene on Xq28, which encodes for a peroxisomal membrane half-transporter of saturated very long chain fatty acids (VLCFA), including C24:0 and C26:0 (Kemp & Wanders, 2010). These mutations prevent VLCFA uptake into peroxisomes for β-oxidation mediated metabolism, causing their accumulation in serum and multiple tissues. The high levels of VLCFAs are regarded as the primary cause of various types of cellular injury, including oxidative stress in the brain and spinal cord in patients. Normalizing lipid metabolism and levels can thus serve to remove the underlying source of injury and prove advantageous for therapy. One approach for this includes bypassing ABCD1 loss through alternative transporters and restoring VLCFA β-oxidation.

Located on the peroxisome membrane, ABCD2 and -3 are members of the same protein family as ABCD1 and are also involved in lipid uptake (Kemp et al., 2011). In 1998, Kemp et al demonstrated that upregulation of ABCD2 correlated with reduced C26:0 levels in patient and Abedi-mutant mouse-derived fibroblasts (Kemp et al., 1998). Moreover, overexpression of ABCD2 or ABCD3 normalized C24:0 β-oxidation in patient fibroblasts (Kemp et al., 1998). Similar results were reported by other groups elsewhere (Netik et al., 1999). In 2004, Pujol et al revealed that Abcd1/Abcd2 double knockout mice have worse clinical and histopathological outcomes than Abcd1 knockouts (Pujol et al., 2004). Importantly, overexpression of Abcd2 in vivo normalized C26:0 levels in brain and adrenal glands, and reduced spinal cord histopathology markers compared to Abcd1 KO (Pujol et al., 2004). These findings provide a strong premise supporting the idea that ABCD family members share some degree of redundancy in regulating the transportation and subsequent metabolism of VLCFA's. Thus, promoting their activity in X-ALD patients may help restore VLCFA levels and ameliorate disease severity.

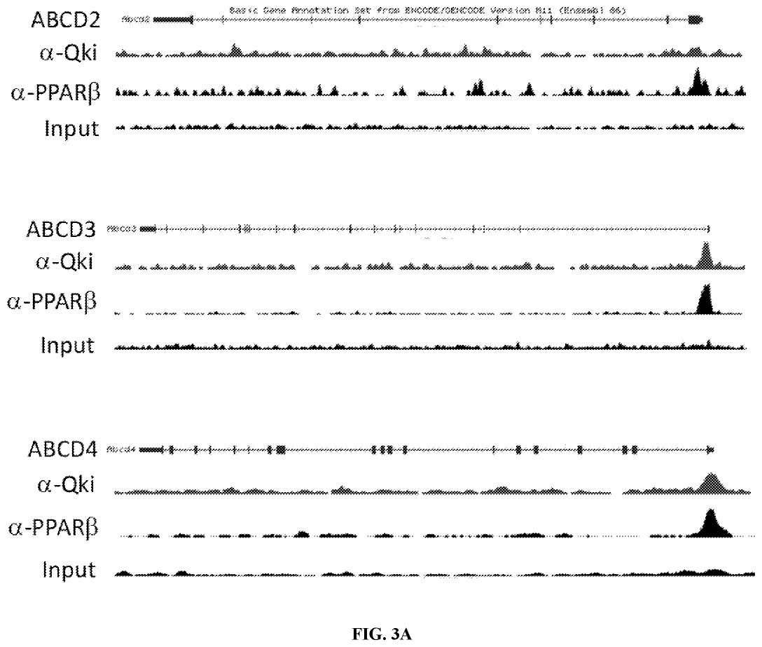

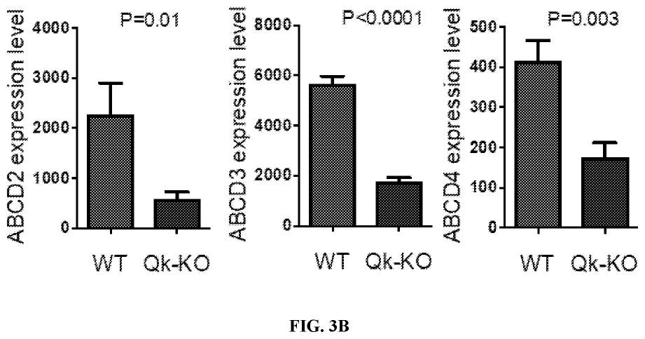

Preliminary findings strongly indicate that PPARβ can likewise modulate ABCD protein expression. ChIP-seq analysis from murine-derived cultured oligodendrocytes, demonstrates PPARβ occupying the promotor regions of Abcd2, -3, and -4 (FIG. 3A). Furthermore, when a critical co-activator (Qki) for PPARβ is deleted, and RNA-seq against WT control performed, there was nearly a 2-fold or higher downregulation of Abcd2, -3, and -4 gene expression in primary and cultured oligodendrocytes (FIG. 3B). In contrast to HSCT, where no clear mechanism is identified and which is restricted to a patient subset, and to previous attempts at targeting the downstream effects of excessive VLCFA's, a new pathway for treatment with two key innovations is provided here. First, by helping to restore peroxisomal transport and β-oxidation of VLCFA's, the upstream event in X-ALD can be directly targeted, which can have broader positive effects on the various forms of injury due to high VLCFA levels. Second, for the same reason as the first, by directly targeting the underlying pathophysiology linking cerebral X-ALD and AMN, the treatment may be applicable to a larger pool of patients.

### Example 3

### Activating Qki-PPARβ-RXRα Treats Chemotherapy-Induced Demyelination

Evidence supporting the central role of demyelination in chemotherapy-induced neurological sequelae has been obtained from brain imaging techniques, such as Diffuse Tenser Imaging, which consistently detect more dramatic changes in the white matter tracts than in the other regions of the brains of patients who have received chemotherapy (Stemmer et al., 1994; Brown et al., 1998; Amidi et al., 2017). The mechanism underlying demyelination in brains exposed to chemotherapy is not clear; however, an extremely puzzling phenomenon might provide some clues. A substantial portion (30%-100%) of patients who undergo long-term evaluation develop delayed-onset adverse neurological sequelae, including demyelination, beginning at least 5 months after the completion of chemotherapy (Stemmer et al., 1994; Brown et al., 1998; Wefel et al., 2010). For instance, in one study, 30% of breast cancer patients in the long-term evaluation group developed delayed-onset cognitive decline at 7.7 months after the completion of chemotherapy (Wefel et al., 2010). Two studies detected white matter changes in up to 70% of breast cancer patients, essentially all of whom developed myelin defects at least 5 months after the cessation of high-dose chemotherapy (Stemmer et al., 1994; Brown et al., 1998). Several lines of evidence suggest that delayed-onset demyelination following chemotherapy is unlikely to be caused by oligodendrocyte death. First, if such demyelination were caused by oligodendrocyte death, it would happen earlier, peaking during or right after chemotherapy. Second, very little oligodendrocyte death has been observed in human or mouse brains exposed to chemotherapy (Han et al., 2008). Third, demyelination can be caused by several different chemotherapeutic drugs with different mechanisms of action, and most of these drugs neither penetrate the brain well nor affect quiescent cells such as oligodendrocytes.

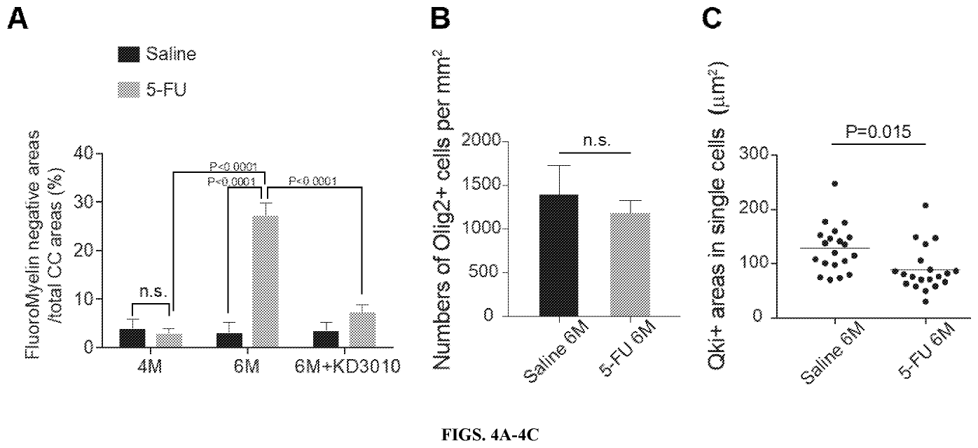

PPARβ and RXRα are nuclear hormone receptors that are easily affected by overall body conditions (Feige et al., 2006; Tyagi et al., 2011; Dreyer et al., 1992; Issemann & Green, 1990). If demyelination in brains exposed to chemotherapy is not primarily caused by oligodendrocyte death, it might be caused by the disruption of nuclear hormone receptor-dependent myelin homeostasis. In determining whether chemotherapy-induced delayed-onset demyelination could be recapitulated in murine models, Han et al found that mice developed delayed-onset demyelination 6 months after receiving a clinically relevant dose of 5-FU for 1 week (Han et al., 2008). The inventors confirmed that mice treated this way (as well as those treated with doxorubicin and methotrexate) exhibited delayed-onset demyelination and myelin lipid reduction in the CC 6 months, but not 4 months, after chemotherapy cessation (FIG. 4A), with minimal changes in myelin protein levels and oligodendrocyte numbers (FIG. 4B). Similar with demyelination induced by defective lipid metabolism (FIG. 2), demyelination induced by 5-FU was also greatly alleviated by RXR/PPARβ agonist bexarotene and KD3010 (FIG. 4A). Suggesting that a defective Qki-PPARβ-RXRα axis causes the reduction of the myelin lipid level in these demyelinating lesions, Qki was greatly reduced in the oligodendrocytes in the lesions (FIG. 4C). These findings suggest that the demyelination induced by chemotherapy and its related neurological sequelae could be caused by defective Qki-PPARβ-RXRα-dependent lipid metabolism and restoring the activity of Qki-PPARβ-RXRα could be effective treatment for chemo brain.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Amidi A, Hosseini S M H, Leemans A, et al. Changes in Brain Structural Networks and Cognitive Functions in Testicular Cancer Patients Receiving Cisplatin-Based Chemotherapy. J Natl Cancer Inst 2017; 109.
Ando S, Tanaka Y, Toyoda Y, Kon K. Turnover of myelin lipids in aging brain. Neurochem Res 2003; 28:5-13.
Armati P J, Mathey E K. The biology of oligodendrocytes. Cambridge; New York: Cambridge University Press; 2010.
Bender C M, Sereika S M, Berga S L, et al. Cognitive impairment associated with adjuvant therapy in breast cancer. Psychooncology 2006; 15:422-30.
Berger J, Forss-Petter S, Eichler F S. Pathophysiology of X-linked adrenoleukodystrophy. Biochimie 2014; 98:135-42.
Bougea A, Voskou P, Kilidireas C, Andreadou E. Capecitabine Induced Multifocal Leukoencephalopathy: Do We Have Always to Switch off the Chemotherapy? Case Rep Oncol Med 2016; 2016:2408269.
Brown M S, Stemmer S M, Simon J H, et al. White matter disease induced by high-dose chemotherapy: longitudinal study with MR imaging and proton spectroscopy. AJNR Am J Neuroradiol 1998; 19:217-21.
Bruck W, Heise E, Friede R L. Leukoencephalopathy after cisplatin therapy. Clin Neuropathol 1989; 8:263-5.
Bruck W, Schmied M, Suchanek G, et al. Oligodendrocytes in the early course of multiple sclerosis. Ann Neurol 1994; 35:65-73.
Castellon S A, Silverman D H, Ganz P A. Breast cancer treatment and cognitive functioning: current status and future challenges in assessment. Breast Cancer Res Treat 2005; 92:199-206.
Choi S M, Lee S H, Yang Y S, Kim B C, Kim M K, Cho K H. 5-fluorouracil-induced leukoencephalopathy in patients with breast cancer. J Korean Med Sci 2001; 16:328-34.
Cramer P E, Cirrito J R, Wesson D W, et al. ApoE-directed therapeutics rapidly clear beta-amyloid and reverse deficits in AD mouse models. Science 2012; 335:1503-6.
Dickey A S, Pineda V V, Tsunemi T, et al. PPAR-delta is repressed in Huntington's disease, is required for normal neuronal function and can be targeted therapeutically. Nat Med 2016; 22:37-45.
Dietrich J. Chemotherapy associated central nervous system damage. Adv Exp Med Biol 2010; 678:77-85.
Downie F P, Mar Fan H G, Houede-Tchen N, Yi Q, Tannock I F. Cognitive function, fatigue, and menopausal symptoms in breast cancer patients receiving adjuvant chemotherapy: evaluation with patient interview after formal assessment. Psychooncology 2006; 15:921-30.
Dreyer C, Krey G, Keller H, Givel F, Helftenbein G, Wahli W. Control of the peroxisomal beta-oxidation pathway by a novel family of nuclear hormone receptors. Cell 1992; 68:879-87.
Eberling J L, Wu C, Tong-Tumbeaugh R, Jagust W J. Estrogen- and tamoxifen-associated effects on brain structure and function. Neuroimage 2004; 21:364-71.
Falleti M G, Sanfilippo A, Maruff P, Weih L, Phillips K A. The nature and severity of cognitive impairment associated with adjuvant chemotherapy in women with breast cancer: a meta-analysis of the current literature. Brain Cogn 2005; 59:60-70.
Fan H G, Houede-Tchen N, Yi Q L, et al. Fatigue, menopausal symptoms, and cognitive function in women after adjuvant chemotherapy for breast cancer: 1- and 2-year follow-up of a prospective controlled study. J Clin Oncol 2005; 23:8025-32.
Feige J N, Gelman L, Michalik L, Desvergne B, Wahli W. From molecular action to physiological outputs: peroxisome proliferator-activated receptors are nuclear receptors at the crossroads of key cellular functions. Prog Lipid Res 2006; 45:120-59.
Filippi M, Rocca M A, Martino G, Horsfield M A, Corni G. Magnetization transfer changes in the normal appearing white matter precede the appearance of enhancing lesions in patients with multiple sclerosis. Ann Neurol 1998; 43:809-14.
Han M H, Lundgren D H, Jaiswal S, et al. Janus-like opposing roles of CD47 in autoimmune brain inflammation in humans and mice. Journal of Experimental Medicine 2012:jem. 20101974.
Han R, Yang Y M, Dietrich J, Luebke A, Mayer-Proschel M, Noble M. Systemic 5-fluorouracil treatment causes a syndrome of delayed myelin destruction in the central nervous system. J Biol 2008; 7:12.
Haykin M E, Gorman M, van Hoff J, Fulbright R K, Baehring J M. Diffusion-weighted MRI correlates of subacute methotrexate-related neurotoxicity. J Neurooncol 2006; 76:153-7.
Hurria A, Rosen C, Rudis C, et al. Cognitive function of older patients receiving adjuvant chemotherapy for breast cancer: a pilot prospective longitudinal study. J Am Geriatr Soc 2006; 54:925-31.
Inagaki M, Yoshikawa E, Matsuoka Y, et al. Smaller regional volumes of brain gray and white matter demonstrated in breast cancer survivors exposed to adjuvant chemotherapy. Cancer 2007; 109:146-56.
Issemann I, Green S. Activation of a member of the steroid hormone receptor superfamily by peroxisome proliferators. Nature 1990; 347:645-50.
Janelsins M C, Heckler C E, Peppone L J, et al. Longitudinal Trajectory and Characterization of Cancer-Related Cognitive Impairment in a Nationwide Cohort Study. J Clin Oncol 2018:JC02018786624.
Janelsins M C, Kohli S, Mobile S G, Usuki K, Ahles T A, Morrow G R. An update on cancer- and chemotherapy-related cognitive dysfunction: current status. Semin Oncol 2011; 38:431-8.
Jenkins V, Shilling V, Deutsch G, et al. A 3-year prospective study of the effects of adjuvant treatments on cognition in women with early stage breast cancer. Br J Cancer 2006; 94:828-34.
Jenkins V, Beveridge H, Low R, Mitra S. Atypical hearing loss in women with breast cancer receiving adjuvant treatment. Breast 2006; 15:448-51.
Johnson M R, Hageboutros A, Wang K, High L, Smith J B, Diasio R B. Life-threatening toxicity in a dihydropyrimidine dehydrogenase-deficient patient after treatment with topical 5-fluorouracil. Clin Cancer Res 1999; 5:2006-11.
Kemp S, Wanders R. Biochemical aspects of X-linked adrenoleukodystrophy. Brain Pathol 2010; 20:831-7.
Kemp S, Theodoulou F L, Wanders R J. Mammalian peroxisomal ABC transporters: from endogenous substrates to pathology and clinical significance. Br J Pharmacol 2011; 164:1753-66.
Kemp S, Wei H M, Lu J F, et al. Gene redundancy and pharmacological gene therapy: implications for X-linked adrenoleukodystrophy. Nat Med 1998; 4:1261-8.
Kleinschmidt-DeMasters B K. Intracarotid BCNU leukoencephalopathy. Cancer 1986; 57:1276-80.
Kreukels B P, Schagen S B, Ridderinkhof K R, Boogerd W, Hamburger H L, van Dam F S. Electrophysiological correlates of information processing in breast-cancer patients treated with adjuvant chemotherapy. Breast Cancer Res Treat 2005; 94:53-61.
Kovalchuk A, Kolb B. Chemo brain: From discerning mechanisms to lifting the brain fog-An aging connection. Cell Cycle 2017; 16:1345-9.
Lassmann H, Bruck W, Lucchinetti C. Heterogeneity of multiple sclerosis pathogenesis: implications for diagnosis and therapy. Trends Mol Med 2001; 7:115-21.
Laule C, Pavlova V, Leung E, et al. Diffusely abnormal white matter in multiple sclerosis: further histologic studies provide evidence for a primary lipid abnormality with neurodegeneration. J Neuropathol Exp Neurol 2013; 72:42-52.
Lucchinetti C, Bruck W, Parisi J, Scheithauer B, Rodriguez M, Lassmann H. Heterogeneity of multiple sclerosis lesions: implications for the pathogenesis of demyelination. Ann Neurol 2000; 47:707-17.
Lucchinetti C F, Bruck W, Rodriguez M, Lassmann H. Distinct patterns of multiple sclerosis pathology indicates heterogeneity on pathogenesis. Brain Pathol 1996; 6:259-74.
Lucchinetti C, Bruck W, Parisi J, Scheithauer B, Rodriguez M, Lassmann H. A quantitative analysis of oligodendrocytes in multiple sclerosis lesions. A study of 113 cases. Brain 1999; 122 (Pt 12):2279-95.
Mahmood A, Raymond G V, Dubey P, Peters C, Moser H W. Survival analysis ofhaematopoietic cell transplantation for childhood cerebral X-linked adrenoleukodystrophy: a comparison study. Lancet Neurol 2007; 6:687-92.
Marcus J, Honigbaum S, Shroff S, Honke K, Rosenbluth J, Dupree J L. Sulfatide is essential for the maintenance of CNS myelin and axon structure. Glia 2006; 53:372-81.
McFarland H F, Martin R. Multiple sclerosis: a complicated picture of autoimmunity. Nat Immunol 2007; 8:913-9.
Meyers C A, Abbruzzese J L. Cognitive functioning in cancer patients: effect of previous treatment. Neurology 1992; 42:434-6.
Miller W P, Rothman S M, Nascene D, et al. Outcomes after allogeneic hematopoietic cell transplantation for childhood cerebral adrenoleukodystrophy: the largest single-institution cohort report. Blood 2011; 118:1971-8.
Moore G R, Laule C, Mackay A, et al. Dirty-appearing white matter in multiple sclerosis: preliminary observations of myelin phospholipid and axonal loss. J Neurol 2008; 255:1802-11.
Morell P, Ousley A H. Metabolic turnover of myelin glycerophospholipids. Neurochem Res 1994; 19:967-74.
Netik A, Forss-Petter S, Holzinger A, Molzer B, Unterrainer G, Berger J. Adrenoleukodystrophy-related protein can compensate functionally for adrenoleukodystrophy protein deficiency (X-ALD): implications for therapy. Hum Mol Genet 1999; 8:907-13.
Noseworthy J H, Lucchinetti C, Rodriguez M, Weinshenker B G. Multiple sclerosis. N Engl J Med 2000; 343:938-52.
Paganini-Hill A, Clark L J. Preliminary assessment of cognitive function in breast cancer patients treated with tamoxifen. Breast Cancer Res Treat 2000; 64:165-76.
Peters C, Charnas L R, Tan Y, et al. Cerebral X-linked adrenoleukodystrophy: the international hematopoietic cell transplantation experience from 1982 to 1999. Blood 2004; 104:881-8.
Popescu B F, Pirko I, Lucchinetti C F. Pathology of multiple sclerosis: where do we stand? Continuum (Minneap Minn) 2013; 19:901-21.
Poppelreuter M, Weis J, Kuiz A K, Tucha 0, Lange K W, Bartsch H H. Cognitive dysfunction and subjective complaints of cancer patients. a cross-sectional study in a cancer rehabilitation centre. Eur J Cancer 2004; 40:43-9.
Price J C, Guan S, Burlingame A, Prusiner S B, Ghaemmaghami S. Analysis of proteome dynamics in the mouse brain. Proc Natl Acad Sci USA 2010; 107:14508-13.
Pujol A, Ferrer I, Camps C, et al. Functional overlap between ABCDI (ALD) and ABCD2 (ALDR) transporters: a therapeutic target for X-adrenoleukodystrophy. Hum Mol Genet 2004; 13:2997-3006.
Raine C S, Scheinberg L, Waltz J M. Multiple sclerosis. Oligodendrocyte survival and proliferation in an active established lesion. Lab Invest 1981; 45:534-46.
Schagen S B, van Dam F S, Muller M J, Boogerd W, Lindeboom J, Bruning P F. Cognitive deficits after postoperative adjuvant chemotherapy for breast carcinoma. Cancer 1999; 85:640-50.
Shilling V, Jenkins V, Fallowfield L, Howell T. The effects of hormone therapy on cognition in breast cancer. J Steroid Biochem Mol Biol 2003; 86:405-12.
Shuper A, Stark B, Kornreich L, et al. Methotrexate treatment protocols and the central nervous system: significant cure with significant neurotoxicity. J Child Neurol 2000; 15:573-80.
Shuper A, Stark B, Kornreich L, Cohen I J, Avrahami G, Yaniv I. Methotrexate-related neurotoxicity in the treatment of childhood acute lymphoblastic leukemia. Isr Med Assoc J 2002; 4:1050-3.
Siegel G J. Basic neurochemistry: molecular, cellular, and medical aspects. 7th ed. Amsterdam; Boston: Elsevier; 2006.
Silverman D H, Dy C J, Castellon S A, et al. Altered frontocortical, cerebellar, and basal ganglia activity in adjuvant-treated breast cancer survivors 5-10 years after chemotherapy. Breast Cancer Res Treat 2007; 103:303-11.
Smith M E, Eng L F. The turnover of the lipid components of myelin. J Am Oil Chem Soc 1965; 42:1013-8.
Smith M E. The turnover of myelin in the adult rat. Biochim Biophys Acta 1968; 164:285-93.
Stemmer S M, Stears J C, Burton B S, Jones R B, Simon J H. White matter changes in patients with breast cancer treated with high-dose chemotherapy and autologous bone marrow support. AJNR Am J Neuroradiol 1994; 15:1267-73.
Stewart A, Collins B, Mackenzie J, Tomiak E, Verma S, Bielajew C. The cognitive effects of adjuvant chemotherapy in early stage breast cancer: a prospective study. Psychooncology 2008; 17:122-30.
Stys P K, Zamponi G W, van Minnen J, Geurts J J. Will the real multiple sclerosis please stand up? Nat Rev Neurosci 2012; 13:507-14.
Toyama B H, Savas J N, Park S K, et al. Identification of long-lived proteins reveals exceptional stability of essential cellular structures. Cell 2013; 154:971-82.
Traka M, Podojil J R, McCarthy D P, Miller S D, Popko B. Oligodendrocyte death results in immune-mediated CNS demyelination. Nat Neurosci 2016; 19:65-74.
Traka M, Podojil J R, McCarthy D P, Miller S D, Popko B. Oligodendrocyte death results in immune-mediated CNS demyelination. Nat Neurosci 2016; 19:65-74.
Tyagi S, Gupta P, Saini A S, Kaushal C, Sharma S. The peroxisome proliferator-activated receptor: A family of nuclear receptors role in various diseases. J Adv Pharm Technol Res 2011; 2:236-40.
Uzman B G, Hedley-Whyte T. Myelin: dynamic or stable? J Gen Physiol 1968; 51:Suppl:8S+.
van der Knaap M S, Bugiani M. Leukodystrophies: a proposed classification system based on pathological changes and pathogenetic mechanisms. Acta Neuropathol 2017; 134:351-82.
Waldman A T. Leukodystrophies. Continuum (Minneap Minn) 2018; 24:130-49.
Wefel J S, Saleeba A K, Buzdar A U, Meyers C A. Acute and late onset cognitive dysfunction associated with chemotherapy in women with breast cancer. Cancer 2010; 116:3348-56.

What is claimed is:

1. A method of treating a dysmyelinating/demyelinating disease or condition in a patient in need thereof, the method comprising administering a therapeutically effective amount of a PPARβ agonist or an RXR agonist to the patient.

2. The method of claim 1, wherein the demyelinating disease is multiple sclerosis.

3. The method of claim 2, wherein the multiple sclerosis is progressive multiple sclerosis.

4. The method of claim 2 or 3, wherein the multiple sclerosis is associated with diffusely abnormal white matter (DAWM).

5. The method of claim 2 or 3, wherein the patient is maintained on a high fat diet.

6. The method of claim 1, wherein the dysmyelinating/demyelinating disease is X-linked adrenoleukodystrophy (X-ALD).

7. The method of claim 1, further comprising performing hematopoietic bone marrow transplantation on the patient.

8. The method of claim 1, wherein the demyelinating disease is adrenomyelopathy (AMN).

9. The method of claim 1, wherein the demyelinating condition is chemotherapy-induced.

10. The method of claim 1, wherein the patient has received chemotherapy.

11. The method of claim 1, wherein the demyelinating condition is chemotherapy-induced cognitive dysfunction.

12. The method of any one of claims 1-11, wherein the method reduces the severity or delays the onset of one or more neurological deficits in the patient.

13. The method of any one of claims 1-11, wherein the method prolongs the survival time of the patient.

14. The method of claim 1, wherein the dysmyelinating/demyelinating disease or condition is a condition of the central nervous system.

15. The method of claim 14, wherein the PPARβ agonist or RXR agonist is able to cross the blood brain barrier.

16. The method of claim 1, wherein the PPARβ agonist or RXR agonist is able to penetrate the peripheral nervous system.

17. The method of any one of claims 1-16, wherein the PPARβ agonist is KD3010, GW0742, Telmisartan, L-165041, Elafibranor, or Seladelpar.

18. The method of any one of claims 1-16, wherein the RXR agonist is bexarotene, LG100268, 13-cis retinoic acid (RA), LG101506, or AGN194204.

19. The method of any one of claims 1-18, wherein the RXR agonist is a RXRα agonist.

20. The method of any one of claims 1-18, wherein demyelination of a nerve axon is suppressed.

21. The method of any one of claims 1-18, wherein demyelination of a nerve axon is reversed.

22. The method of any one of claims 1-18, wherein the method increases the level of myelin lipids in the patient.

23. The method of any one of claims 1-22, wherein the method restores Qki-PPARβ-RXRα-dependent lipid metabolism in myelin.

24. Use of a composition comprising a therapeutically effective amount of a PPARβ agonist or an RXR agonist in the preparation of a medicament for the treatment of a dysmyelinating/demyelinating disease.

25. A composition comprising a therapeutically effective amount of a PPARβ agonist or an RXR agonist for use in treating a dysmyelinating/demyelinating disease.
